(19) United States
(12) Patent Application Publication
(10) Pub. No.: US 2025/0367156 A1
(43) Pub. Date: Dec. 4, 2025

(54) FOOD COMPOSITION IN A DRY POWDER FORM FOR ENDURANCE TRAINING-RACING, AND METABOLIC EFFICIENCY

(71) Applicant: Leighton Owen Phillips, Holly Springs, NC (US)

(72) Inventor: Leighton Owen Phillips, Holly Springs, NC (US)

(21) Appl. No.: 18/732,700

(22) Filed: Jun. 4, 2024

(51) Int. Cl. A61K 31/20; A61K 31/197; A61K 31/225

(52) U.S. Cl. CPC A61K 31/20; A61K 31/197; A61K 31/225

(57) ABSTRACT

Disclosed is a dehydrated blend of C8-C10 Medium Chain Triglycerides and the amino acid Glutamine. The present invention provides either a full formulation (zone 2, aerobic intensity) in its own right or a partial substitution (zone 3-4, higher intensity) for carbohydrates in fueling formulations or an additional fuel substrate alongside carbohydrates (zone 4-5, high-intensity training-racing) in such formulations. The product serves as a food ingredient in endurance fueling products like drinks, gels, powders, bars, gummies, etc., promoting enhanced fuel-substrate oxidation to support training-racing intensities while mitigating well-known risks associated with high carbohydrate fueling. These risks include gut/GI distress, reduced aerobic training adaptations, reduced lipolysis, fat oxidation, reduced glycogen retention, elevated lactate production, and chronically heightened blood-sugar levels.

### FIELD OF THE INVENTION

The present invention relates to extended substrate ingredients for endurance training and racing, in particular to a food composition in a dry powder form for endurance training-racing, and general metabolic efficiency.

### BACKGROUND OF THE INVENTION

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

Currently, providing adequate fueling support for endurance training and racing poses several challenges. Endurance activities place significant demands on the body, requiring sustained access to fuel substrates and proper nutrition. Whether derived from within the body (such as muscle-liver glycogen, fats, ketones, and amino acids) or from external sources (such as consumed carbohydrates, fats, and amino acids), the availability of these fuels and substrates is crucial for maintaining performance, preventing fatigue, and facilitating rapid recovery. Competitive athletes can benefit from optimizing their metabolic efficiency and flexibility in accessing and utilizing both internal and external fuel sources and nutrition during training and racing. Furthermore, general active-inactive individuals would benefit from improved glucose-insulin homeostasis, and metabolic efficiency.

Chinese patent CN110810845A filed by ZHANG JIE et.al. talks about whole-nutrition formula powder with ketone-producing, fat-reducing, and sugar-resisting functions and the application of whole-nutrition formula powder. The whole-nutrition formula powder is prepared from the following raw materials: small molecular collagen peptide, whey protein concentrate powder, raw pressed coconut oil, linseed oil, glutamine, maltodextrin, fructooligosaccharide, resistant dextrin, complex minerals, decavitamin, mono/diglycerol fatty acid ester (emulsifier) and edible essence. The invention further relates to the application of the whole-nutrition formula powder in the preparation of functional food with ketone-producing, fat-reducing, and sugar-resisting functions. The whole-nutrition formula powder has the advantages as follows: the whole-nutrition formula powder is scientific in formula and comprehensive in nutrients, components cover three major macronutrients including carbohydrate, fat, and protein, meanwhile, all vitamins and minerals recommended by Chinese Resident Dietary Guidelines 2016 Version RNI are provided, the characteristics that MCT (medium chain triglyceride) cannot become depot fat, so that energy intake and storage are controlled, and the proposed purpose of controlling the body weight is supported.

Korean patent application KR100526163B1 filed by HAN DAE SEOK et. al. discloses a heavy chain triglycerides (medium chain triglyceride), oktakosanol, and plant extract composition for a physical performance enhancer.

It is important to note that the body has distinct compartmentalized energy systems to support various levels of physical intensity. Fat serves as the predominant energy source for the body, followed by carbohydrates. Typically, as the intensity of physical exercise rises, the body's preference for fuel substrate shifts from primarily using fat to increasingly relying on carbohydrates. Similar to the training of muscles and respiratory fitness, the metabolic efficiency and flexibility of these energy systems can be significantly enhanced (our lab's data has recorded cases, of up to three times baseline values) through physical training, proper substrate-fuel nutrition choice before-during training, and from everyday diet.

However, overconsumption and the predominant reliance on carbohydrates, particularly during training and racing, have been shown to lead to inefficiencies in an athlete's fat oxidation metabolism and a blunting of key aerobic training adaptations. This can result in accelerated depletion of muscle and liver glycogen stores during training-racing, further reinforcing dependency on carbohydrate intake during training and racing.

The overconsumption of carbohydrates in athletes has been extensively studied and shown to be a key causative factor behind various acute and chronic issues: 1) Acute gut/GI distress, such as nausea, cramping, and diarrhea, is commonly observed in endurance sports and remains a major cause of DNF (Do Not Finish) race outcomes, and in severe cases, hospitalization. 2) Acute elevation in lactate (and ammonia) levels, along with higher inflammatory markers during exercise, contribute to faster onset of fatigue, compromised muscular contractions, and damage to muscle and gut membrane tissues. 3) Chronically elevated blood glucose levels (and resting lactate levels) serve as hallmark precursors to chronic diseases such as diabetes, and associated complications including cardiovascular disease.

To tackle these challenges, endurance athletes frequently experiment with various fuel combinations, most of which are predominantly carbohydrate-based. They then turn to sports nutritionists and dietitians for further evaluation and alternative recommendations. However, this specification acknowledges the necessity for diverse fuel-substrate compositions tailored to different exercise intensities during endurance training and racing. This approach aims to lessen reliance on carbohydrate-dominated fuels, whether endogenous or exogenous while accentuating the efficiency of aerobic adaptations, fat breakdown (lipolysis), fat oxidation, carbohydrate gut-transit times, and lactate efficiency.

The disadvantages and limitations of traditional approaches will become apparent to the person skilled in the art through a comparison of the described method with some aspects of the present disclosure, as put forward in the remainder of the present application.

### SUMMARY OF INVENTION

The present invention mainly cures and solves the technical problems existing in the prior art. In response to these problems, the present invention provides an extended substrate ingredient, to enable improved endogenous fat-oxidation efficiency and thereby, glycogen and amino acid retention, for improved fueling and recovery nutrition in endurance training and racing.

An aspect of the present invention relates to a food composition for endurance training-racing and metabolic-efficiency. The food composition includes medium-chain triglycerides (MCTs); and a glutamine amino acid. The medium-chain triglycerides (MCTs) and the glutamine amino acid are fused to obtain the food-nutrition composition in a dry powder form.

In an aspect, the MCTs are a plurality of fatty acids with aliphatic tails containing six to twelve carbon atoms.

In an aspect, the fatty acids include octanoic acid (CS); and decanoic acid (C10).

In an aspect, the six to twelve carbon atoms are medium-chain fatty acids (MCFAs).

In an aspect, the medium-chain triglycerides (MCTs) are present in a concentration ranging from 45% to 50% by weight of the total food composition.

In an aspect, the glutamine amino acid is present in concentrations ranging from 35% to 45% by weight of the total food composition.

In an aspect, the food composition further includes residual water/moisture, and minor fragments of MCT (excluding C8/C10) are present in the concentration, ranging from 5-15% by weight of the total food composition.

In an aspect, the dry powder form is suitable for mixing with water or a plurality of beverages, gels, gummies, bars, or capsule-tablet form factors for consumption.

In an aspect, the food composition is water-dissolvable.

In an aspect, the food composition is a non-carbohydrate tasteless food compound.

In an aspect, the food composition is a fuel substrate for aerobic oxidation.

Another aspect of the present disclosure relates to a food composition in a dry powder form to support fueling and recovery nutrition for endurance training and racing intensities. The food composition comprises a fusion of C8-C10 medium-chain triglycerides (MCT) and the amino acid glutamine. To date, MCT has been commercially available in both fluid (oil) and powder form. Fluid formats are dosage and utility limited, for use in sports nutrition on account of fluid-oil compliance (nausea, belching, etc.) issues, and also from practical mobility-compact storage issues. Powder-based forms are typically spray/freeze-dried to low-cost carbohydrates like maltodextrins (glucose polymer) which if used would only further contribute to the carbohydrate load issues noted in the above background section of this application. Powder-based forms of MCT can also be spray-dried onto collagen peptides.

Collagen, however, has no functional role in improving metabolic efficiency in endurance training-racing, and at such small doses would not deliver any other functional purpose.

In an aspect, the food ingredient used in endurance fueling products would support broader types, and higher levels, of fuel-substrates (both endogenous fat/glycogen, and exogenous fat/carbohydrate) oxidation in supporting training-racing intensities, while avoiding known risks of consistent higher carbohydrate fueling, those being—blunted lipolysis and fat-oxidation, accelerated depletion of tissue glycogen/amino stores, gut/GI distress, heightened lactate levels, and in time chronically heightened blood-sugar levels. Examples of endurance fueling products and formats included, but are not limited to drinks, gels, powders, bars, and gummies.

Other features of embodiments of the present disclosure will be apparent from the accompanying drawings and from the detailed description that follows.

### BRIEF DESCRIPTION OF FIGURES

In the figures, similar components and/or features may have the same reference label. Further, various components of the same type may be distinguished by following the reference label with a second label that distinguishes among the similar components. If only the first reference label is used in the specification, the description applies to any one of the similar components having the same first reference label irrespective of the second reference label.

Embodiments of the present disclosure will be described herein by way of example with reference to the accompanying figures, in which:

FIG. 1 illustrates an exemplary view of medium-chain triglycerides (C8 and C10 noted here) according to an embodiment of the present disclosure.

FIG. 2 illustrates an exemplary view of a skeletal formula of Glutamine (practical application would be isomer form, L-Glutamine) according to an embodiment of the present disclosure.

### DETAILED DESCRIPTION OF THE ILLUSTRATIVE EMBODIMENTS

The following detailed description is made with reference to the accompanying figures.

It is further important to note that the figures included in the present disclosure are not to scale. While the figures are intended to illustrate the key features and functionality of the invention, they are not intended to represent the size or proportion of any various components accurately. Instead, the figures are intended to provide a clear and concise depiction of the invention that will aid in understanding its operation and functionality. It should be understood that the relative sizes and dimensions of any components may differ from what is shown in the figures and that the figures should not be relied upon for precise measurements or scaling. The description provided herein should be consulted for further details regarding the size and dimensions of the invention.

It is important to note that certain aspects of the present disclosure may not be explicitly described herein. However, it is assumed that these aspects follow a common technical knowledge that is widely known to an ordinary person skilled in the art. As such, it is not necessary to provide explicit details regarding these aspects in this disclosure. The skilled person would be expected to understand and implement these aspects based on their general knowledge and expertise. The purpose of the present disclosure is to provide a comprehensive and clear description of the invention while acknowledging that certain aspects may be considered implicit to those skilled in the art.

The present invention discloses a food composition for endurance training-racing and general metabolic-efficiency. The food composition includes medium-chain triglycerides (MCTs); and a glutamine amino acid. The medium-chain triglycerides (MCTs) and the glutamine amino acid are fused to obtain the food-nutrition composition in a dry powder form. In an embodiment, the MCTs are a plurality of fatty acids with aliphatic tails containing six to twelve carbon atoms. Examples of the fatty acids include but are not limited to octanoic acid (CS); and decanoic acid (C10). In an embodiment, the six to twelve carbon atoms are medium-chain fatty acids (MCFAs). In an embodiment, the medium-chain triglycerides (MCTs) are present in a concentration ranging from 45% to 50% by weight of the total food composition.

In an embodiment, the glutamine amino acid is present in concentrations ranging from 35% to 45% by weight of the total food composition. In an embodiment, the food composition further includes residual water/moisture, and minor fragments of MCT (excluding C8/C10) are present in the concentration, ranging from 5-15% by weight of the total food composition. In an embodiment, the dry powder form is suitable for mixing with water or a plurality of beverages, gels, bars, gummies, or capsule-tablet form factors for consumption. In an embodiment, the food composition is water-dissolvable. In an embodiment, the food composition is a non-carbohydrate tasteless food compound. In an embodiment, the food composition is a fuel substrate for aerobic oxidation.

According to an embodiment herein, the nutrients of the present food composition have various advantages such as acting as fuel to the body (like carbs/fats); supporting aerobic development; supporting fat lipolysis and oxidation; supporting glycogen retention, and replenishment; accelerated carbohydrate gut transit, and fuel the gut tissue and, nutrient support for gut membrance integrity and resilience.

Research has demonstrated that a combined nutrient-ingredient can enhance aerobic capacity and efficiency through various mechanisms. These include promoting mitochondrial biogenesis (MCT), increasing lipolysis (MCT/Glutamine), and enhancing fat oxidation (MCT/Glutamine). Additionally, this combination supports glycogen retention and replenishment (Glutamine) while also lowering lactate levels (MCT). These effects collectively contribute to improving overall endurance performance and metabolic efficiency during exercise.

Research has indicated that a combined nutrient-ingredient can accelerate the transit time of carbohydrates through the gut (MVT, and enhance gut membrane integrity (Glutamine). Furthermore, certain nutrients have demonstrated the ability to bolster immune capacity fuel (Glutamine). Additionally, these nutrients have been shown to increase circulating GLP-1, GIP, and insulin concentrations in vivo (Glutamine) which could support improved glycemic homeostasis in recreationally active/inactive individuals. These findings highlight the potential of this combination to support gut health, enhance immune function, and regulate key metabolic hormones, thereby contributing to overall well-being and physiological balance.

The present invention anticipates the utilization of this combined nutrient, as an extended substrate in various scenarios. Firstly, during Zone 2 aerobic intensity training, it will be incorporated into drinks, gels, and gummies. Secondly, in Zone 4 threshold/HIIT and racing scenarios, it will again be consumed in the form of drinks, gels, and gummies. Lastly, during Zone 5+ highest intensity racing, it will be administered as a gel and gummies.

Third-party research and our athlete lab data affirm that using the “RIGHT FUEL-substrates at the RIGHT TIME-intensity” can significantly prolong the duration where fat remains the primary energy source. This strategy effectively preserves glycogen and lowers lactate for consistent pacing and strong finishes during races.

In addition to performance enhancements, athletes who have embraced this approach commonly report reduced Gut/GI distress during high-intensity training/racing, attributed to decreased reliance and intake, on carbohydrates during training-racing, and expedited recovery post workout/race.

According to an embodiment herein, Octanoic Acid (C8) is a saturated fatty acid, also known as Caprylic acid. It is classified as a medium chain triglyceride (MCT) due to its 8 carbon atoms (‘Octa’). Octanoic Acid (C8) is formed as a resultant fatty acid from the conversion of long-chain triglycerides in the cytoplasm. It serves as a fuel substrate for the Citric Acid Cycle (Mitochondrial beta-oxidation) and facilitates passive transport across both gastrointestinal and hepatic membranes. Acyl-carnitine transporters are required in skeletal/cardiac cell membranes for Octanoic Acid (C8) transport. Octanoic Acid (C8) is naturally found in breast milk, coconut, and palm oil.

According to an embodiment herein, Decanoic Acid (C10) is a saturated fatty acid, also known as Capric acid. It is classified as a Medium Chain Triglyceride (MCT) due to its 10 carbon atoms (‘Deca’). Decanoic Acid (C10) is formed as a resultant fatty acid from the conversion of long-chain triglycerides in the cytoplasm. It serves as a fuel substrate for the Citric Acid Cycle (Mitochondrial beta-oxidation) and facilitates passive transport across both gastrointestinal and hepatic membranes. Acyl-carnitine transporters are required in skeletal/cardiac cell membranes for Decanoic Acid (C10) transport. Moreover, it acts as a substrate for hepatic intermediates, leading to the notable production of ketone bodies. Decanoic Acid (C10) also stimulates GLP-1 secretion and enhances glucose tolerance. Naturally, it is found in breast milk and coconut oil

According to an embodiment herein, glutamine is classified as an amino acid, specifically a conditionally essential one, meaning its requirement increases during periods of stress, catabolic conditions, physical exercise, or injury. It holds the distinction of being the most abundant amino acid in the body and is primarily stored in skeletal muscle. Glutamine production primarily occurs in muscle and brain tissues. Functionally, glutamine serves as a fuel substrate for the Citric Acid Cycle (Mitochondrial) and supports protein synthesis. It also acts as an acute clinical supplementation in catabolic disease states, particularly those related to immune and gastrointestinal functions. Additionally, glutamine stimulates GLP-1 secretion. Naturally, glutamine can be found in various food sources such as meats (beef, chicken, fish, etc.), milk, cheese, and eggs.

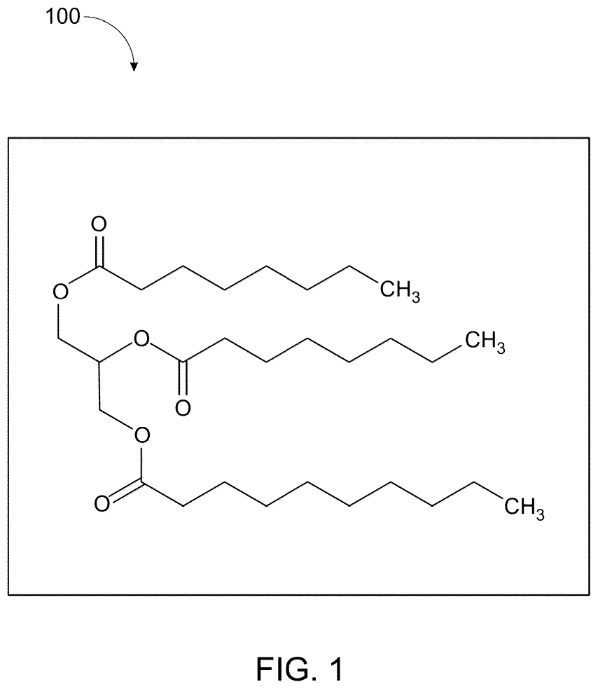

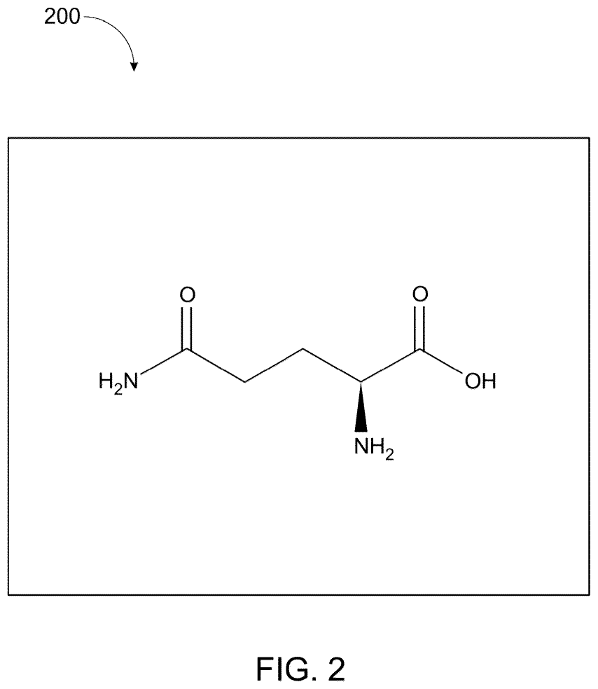

FIG. 1 illustrates an exemplary view 100 of medium-chain triglycerides (C8 and C10 noted here) according to an embodiment of the present disclosure. Typically, MCTs refer to triglycerides that contain two or three fatty acids with aliphatic tails containing 6 to 12 carbon atoms, known as medium-chain fatty acids (MCFAs). Palm kernel oil and coconut oil are abundant sources of MCTs, often used for commercial extraction. MCT due to their lower number of carbon atoms, can traverse both gastrointestinal and intercellular membranes thereby leading to rapid transport and oxidation (as a substrate) in the body. MCTs facilitate the transport of other compounds across the gastrointestinal membrane, thereby increasing absorption rates—this is true for carbohydrates, peptides, and drug compounds. MCTs have also been shown to raise blood-ketone levels, lower blood lactate concentration, and RPE (rate of perceived exertion) during moderate-intensity exercise, and extend duration during high-intensity exercise. MCTs are preferentially oxidized (versus long-chain fats) during high-intensity exercise. MCT has also been shown to increase mitochondrial biogenesis and improve high-temperature-induced exercise performance impairment. FIG. 2 illustrates an exemplary view 200 of the skeletal formula of Glutamine according to an embodiment of the present disclosure. Glutamine is an α-amino acid involved in protein biosynthesis, immune function, and a substrate for cellular energy to multiple tissues of the body. Glutamine supplementation has been shown to improve insulin sensitivity, and fatty-acid utilization (via enhanced mitochondrial respiration). Aside from its well-documented support for gut-tissue integrity issues from exercise, Glutamine has been shown to offer a protective effect against muscle tissue damage markers (i.e., CK, etc.) and inflammatory markers (IL-6, IL10, etc.) from eccentric exercise. Glutamine is suggested as an anti-fatigue amino acid, based on multiple studies highlighting functions of increased glycogen synthesis, and reduced muscle and brain ammonia accumulation.

It is considered non-essential and conditionally essential in humans, as the body can typically produce enough. However, many studies have shown that in periods of stress, or catabolic states (disease, infection, intensive exercise, etc.) its demand increases, requiring supplementary intake. Multiple studies have shown Glutamine (lowered muscle reserve) as an indicator of exercise stress, and overtraining. Glutamine shares a side chain similar to glutamic acid but with an amide replacing the carboxylic acid group. Glutamine is coded by the codons CAA and CAG and is the most abundant free amino acid in human blood. Protein-rich foods like pork, beef, chicken, fish, dairy products, and eggs, as well as various vegetables, wheat, papaya, Brussels sprouts, and fermented foods such as miso, are dietary sources of glutamine.

According to an embodiment herein, the present invention provides a dried powder of fusion of C8-C10 medium chain triglycerides, and the amino acid Glutamine. Further, the invention provides a full (for low-intensity, zone 2, long aerobic training sessions), or partial (for higher-intensity, zone 3-4 tempo training sessions) fuel-substrate replacement in sports fueling formulations, and/or an incremental fuel-substrate to carbohydrates (for racing, high-intensity, zone 4-5 training or racing) in fueling formulations.

In an embodiment, the food composition of the present invention supports fueling for endurance training and racing intensities, while 1) reducing further challenge to gut absorption/gut distress—a major issue in endurance sports; 2) reducing the risks of blunting aerobic training adaptations, lipolysis, and fat-oxidation metabolic processes. Fat-oxidation efficiency (and subsequent glycogen retention) is a key determinate of performance in endurance sports; 3) reducing further heightening of lactate production, and ammonia accumulation—leading to muscle contraction dysfunction—a known factor triggering fatigue in intense physical activity; 4) reducing inflammatory mediator concentration from exercise driven muscle contractions; and, 5) minimizing chronic risk complications (chronically elevated blood glucose, and pre-diabetic states) associated with consistent high carbohydrate consumption.

A novel fuel substrate has been created here by combining particular (C8/C10) medium-chain triglycerides (MCT) and Glutamine. This tasteless food compound possesses unique features, allowing for swift absorption and oxidation, with MCT, a known fuel substrate of low and high-intensity exercise, passing through gut and intra-cellular membranes, without the need for supportive membrane-transporters (like Glut-4 (glucose), Glut-5 (fructose), and CD36 (long-chain fatty acids).

Further, by fusing specific (C8/C10) medium-chain triglycerides (MCT) and Glutamine as a new fuel-substrate, a non-carbohydrate tasteless food compound has been invented-produced here, with the characteristics of, 1) fuel for, and nutritive support to gut membrane integrity, 2) provision of incremental non-carbohydrate substrate—an incremental fuel for muscle/metabolism, 3) little-no effect on raising blood sugar levels, thereby not reducing fat-oxidation—on the contrary increases fat-oxidation, 4) enhances the synthesis of glycogen in muscle tissue, 5) supports the reduced accumulation of ammonia (generated during exercise, triggering fatigue) in muscle and buffered ammonia in the brain, 6) lower rates of perceived exertion.

By incorporating a portion of the novel invention, with carbohydrates into a more comprehensive fueling mixture (or taken alone, without added carbohydrates), the innovative creation aims to reduce the likelihood of Gut/GI distress, maximize fat-oxidation, support glycogen retention, decrease lactate production, buffer ammonia, and mitigate the potential risks of chronic diseases linked to prolonged elevated blood sugar levels, which have become more prevalent in athletes consuming high-carbohydrate diets regularly. The fusion into a dried-powder format would provide a consistent ingredient for endurance fuel products, mixed into drinks, gels, gummies, and bars as typical formats used in endurance sports.

The present disclosure further describes various nutrient research references referred to while producing the food composition for endurance training-racing and metabolic efficiency.

### Fuel-Substrate (Glutamine)

Mol Syst Biol. 2013 December. Glutamine-driven oxidative phosphorylation is a major ATP source in transformed mammalian cells in both normoxia and hypoxia.
Nature Communications. 2019 May 9. Competition for nutrients and its role in controlling immune responses.
National Academies Press, Institute of Medicine (US) Committee on Military Nutrition Research. Washington (DC) 1999. Military Strategies for Sustainment of Nutrition and Immune Function in the Field (Glutamine).
Nutraceutical Research 2013 February. L-glutamine supplementation: Effects on endurance, power, and recovery.
Nutrients. 2019 April. Glutamine as an Anti-Fatigue Amino Acid in Sports Nutrition Experimental & Molecular Medicine, 2020 September. Glutamine reliance in cell metabolism.
Metabolomics, 2023 July. Monitoring and modelling the glutamine metabolic pathway: a review and future perspectives.
Am J Physiol Endocrinol Metab. 2012 August. Whole body and skeletal muscle glutamine metabolism in healthy subjects.
Metabolites. 2019 May. The Fate of Glutamine in Human Metabolism. The Interplay with Glucose in Proliferating Cells.
ResearchGate. 2018 May. Role of glutamine, as free or dipeptide form, on muscle recovery from resistance training: a review study.

### Fuel-Substrate (MCT)

Am J Physiol Gastrointest 2023 October. Medium-chain fatty acid oxidation is independent of I-carnitine in the liver and kidney but not in the heart and skeletal muscle.
J Appl Physiol 1995 September. Metabolic availability of medium-chain triglycerides coingested with carbohydrates during prolonged exercise.
Front Nutr. 2020 January. Medium Chain Triglycerides Modulate the Ketogenic Effect of a Metabolic Switch.
Front Nutr. 2019. Plasma Ketone and Medium Chain Fatty Acid Response in Humans Consuming Different Medium Chain Triglycerides During a Metabolic Study Day.

### Aerobic Development—

### Mitochondrial Biogenesis (MCT/Glutamine):

PLoS One. 2018 February. Medium Chain Triglycerides enhances exercise endurance through the increased mitochondrial biogenesis and metabolism.
J Neurochem. 2014 May, The ketogenic diet component decanoic acid increases mitochondrial citrate synthase and complex 1 activity in neuronal cells.
Nutr Res. 2020 December. Glutamine depletion disrupts mitochondrial integrity and impairs C2C12 myoblast proliferation, differentiation, and the heat-shock response.

### Improved Fat Oxidation (MCT/Glutamine)

MDPI. 2022 September. Cystine/Glutamine Mixture Supplementation Attenuated Fatigue during Endurance Exercise in Healthy Young Men by Enhancing Fatty Acid Utilization.
Int J Obes Relat Metab Disord. 2003 December, Greater rise in fat oxidation with medium-chain triglyceride consumption relative to long-chain triglyceride is associated with lower initial body weight and greater loss of subcutaneous adipose tissue.
Nutrients. 2021 January. Effect of Ingestion of Medium-Chain Triglycerides on Substrate Oxidation during Aerobic Exercise Could Depend on Sex Difference in Middle-Aged Sedentary Persons.

### Increased Lipolysis (MCT)

J Appl Physio. 2003 July. Glutamine supplementation further enhances exercise-induced plasma IL6.
Nutrients. 2022 February. Effect of Continuous Ingestion of 2 g of Medium-Chain Triglycerides on Substrate Metabolism during Low-Intensity Physical Activity.

### Lowered Lactate (MCT)

J Nutr Sci Vitaminol (Tokyo). 2009 April. Effect of ingestion of medium-chain triacylglycerols on moderate- and high-intensity exercise in recreational athletes.

### Glycogen Retention/Recovery (Glutamine/MCT)

J Appl Physiol 1999 June. Effect of oral glutamine on whole body carbohydrate storage during recovery from exhaustive exercise.
J Nutr Sci Vitaminol (Tokyo). 2008 December. Effects of dietary medium-chain triacylglycerol on mRNA level of gluconeogenic enzymes in malnourished rats.

### Gut Membrane Integrity (Glutamine)

J Epithel Biol Pharmacol. 2012 January. Role of Glutamine in Protection of Intestinal Epithelial Tight Junctions.
Eur J Nutr. 2022 June. Effects of oral cystine and glutamine on exercise-induced changes in gastrointestinal permeability and damage markers in young men.
PloS One. 2022 Apr. 13. A systematic review: Role of dietary supplements on markers of exercise-associated gut damage and permeability.

### Immune Health (Glutamine)

Nat Commun. 2019 May, Competition for nutrients and its role in controlling immune responses.
Nutrients. 2018 November. Glutamine: Metabolism and Immune Function, Supplementation and Clinical Translation.
Proc Nutr Soc. 2023 February. Glutamine metabolism and optimal immune and CNS function.

### Muscle Tissue Recovery (Glutamine)

Front Physiol. 2023 April. L-glutamine is better for treatment than prevention in exhaustive exercise.

### Increased IL-6 Myokine Secretion (Glutamine):

Proc Nutr Soc. 2004 May. The metabolic role of IL-6 produced during exercise: is IL-6 an exercise factor?
J Appl Physiol 2003 February. Glutamine supplementation further enhances exercise-induced plasma 11-6.
FASEB J. 2001 December. Transcriptional activation of the IL-6 gene in human contracting skeletal muscle: influence of muscle glycogen content,

### GLP-1/Glucose Tolerance

Am J Clin Nutr. 2009 January. Oral Glutamine Increases Circulating GLP-1, Glucagon and Insulin Levels in Lean, Obese, and Type 2 Diabetic Subjects.
Front. Nutr., 24 Mar. 2022. Dietary Medium-Chain Triglyceride Decanoate Affects Glucose Homeostasis Through GPR84-Mediated GLP-1 Secretion in Mice.

### Glucose/Fructose (Sucrose) Anti-Aerobic/Aerobic Blunting:

### Blunt Fat-Oxidation (Exercise/Skeletal Muscle):

Am J Physiol. 1997 August. Fatty acid oxidation is directly regulated by carbohydrate metabolism during exercise.
Am J Physiol. 1997 October. Lipolytic suppression following carbohydrate ingestion limits fat oxidation during exercise.

### Blunt Fat-Oxidation (Systemic):

J Nutr Biochem. 2023 April. Fructose impairs fat oxidation: Implications for the mechanism of western diet-induced NAFLD.
Br J Nutr. 2008 August. A high-fructose diet impairs basal and stress-mediated lipid metabolism in healthy male subjects.

### Avoid Mitochondria Enzyme Blunting:

Genes Nutr. 2017 December. Fructose ingestion impairs the expression of genes involved in skeletal muscle's adaptive response to aerobic exercise.
Diabetes Metab. 2008 February. Effects of a four-week high-fructose diet on gene expression in skeletal muscle of healthy men.
Apoptosis. 2015 July. Fructose induces mitochondrial dysfunction and triggers apoptosis in skeletal muscle cells by provoking oxidative stress.
Nutrients. 2017 April. Fructose-Rich Diet Affects Mitochondrial DNA Damage and Repair in Rats.

### Avoid Glut4 Transporter Blunting:

Diabetes Metab. 2008 February. Effects of a four-week high-fructose diet on gene expression in skeletal muscle of healthy men.
Am J Physiol Endocrinol Metab. 2014 February. Suppression of the GLUT4 adaptive response to exercise in fructose-fed rats.
Genes Nutr. 2017 December. Fructose ingestion impairs the expression of genes involved in skeletal muscle's adaptive response to aerobic exercise.
Physiol Rev. 2013 July. Exercise, GLUT4, and skeletal muscle glucose uptake.

### Fructose—Gut Membrane/Permeability:

Hepatology, 2021 June, Fructose Promotes Leaky Gut, Endotoxemia, and Liver Fibrosis through CYP2E1-Mediated Oxidative and Nitrative Stress.
Cell Mol Gastroenterol Hepatol. 2020 September. Dietary Fructose Alters the Composition, Localization, and Metabolism of Gut Microbiota in Association With Worsening Colitis.
Nutrients. 2020 November. Fructose-Induced Intestinal Microbiota Shift Following Two Types of Short-Term High-Fructose Dietary Phases,

### Fructose—Systemic Metabolic Conditions:

Nutrients. 2017 March. High Dietary Fructose: Direct or Indirect Dangerous Factors Disturbing Tissue and Organ Functions.
Hum Immunol. 2022 June. High fructose diet: A risk factor for immune system dysregulation.
J Clin Endocrinol Metab. 2021 October. Consuming Sucrose- or HFCS-sweetened Beverages Increases Hepatic Lipid and Decreases Insulin Sensitivity in Adults.
J Nutr. 2012 February. Greater fructose consumption is associated with cardiometabolic risk markers and visceral adiposity in adolescents.

Modifications and variations of the compositions and methods described herein will be obvious to those skilled in the art from the foregoing description. Such modifications and variations are intended to come within the scope of the appended claims.

That which is claimed is:

1. A food composition for endurance training-racing and metabolic-efficiency, comprising. a plurality of medium-chain triglycerides (MCTs); and
a glutamine amino acid, wherein the plurality of medium-chain triglycerides (MCTs) and the glutamine amino acid fused to obtain the food-nutrition composition in a dry powder form.

2. The food composition according to claim 1, wherein the MCTs are a plurality of fatty acids with aliphatic tails containing six to twelve carbon atoms.

3. The food composition according to claim 2, wherein the plurality of fatty acids comprises: octanoic acid (C8); and decanoic acid (C10).

4. The food composition according to claim 2, wherein the six to twelve carbon atoms are medium-chain fatty acids (MCFAs).

5. The food composition according to claim 1, wherein the plurality of medium-chain triglycerides (MCTs) are present in a concentration ranging from 45% to 50% by weight of the total food composition.

6. The food composition according to claim 1, wherein the glutamine amino acid is present in the concentration ranging from 35% to 45% by weight of the total food composition.

7. The food composition according to claim 1, further comprises residual water or moisture, and minor fragments of MCT (excluding C8/C10) are present in the concentration, ranging from 5-15% by weight of the total food composition.

8. The food composition according to claim 1, wherein the dry powder form is suitable for mixing with water or a plurality of beverages, gels, gummies, bars, or capsule-tablet form-factors for consumption.

9. The food composition according to claim 1, wherein the food composition is water-dissolvable.

10. The food composition according to claim 1, wherein the food composition is a non-carbohydrate tasteless food compound.

11. The food composition according to claim 1, wherein the food composition is a fuel substrate for aerobic oxidation.
